(19) United States
(12) Patent Application Publication
(10) Pub. No.: US 2019/0211150 A1
(43) Pub. Date: Jul. 11, 2019

(54) PEGYLATED RESOLVING AGENTS FOR IMPROVED RESOLUTION OF RACEMIC MIXTURE

(71) Applicant: Khashayar KARIMIAN, Tehran (IR)

(72) Inventors: Khashayar KARIMIAN, TEHRAN (IR); Javad MOKHTARI ALIABAD, TEHRAN (IR); Sahar AZARNOOSH, TEHRAN (IR)

(21) Appl. No.: 16/326,924

(22) PCT Filed: Aug. 22, 2017
(86) PCT No.: PCT/IB2017/055057

§ 371 Date: Feb. 21, 2019

(30) Foreign Application Priority Data
Aug. 31, 2016 (IR) 13955014000300700

(51) Int. Cl. C08G 65/323; C08G 65/332; C08G 65/333; C07C 227/34

(52) U.S. Cl. CPC C08G 65/323; C08G 65/3326; C07B 2200/07; C07C 227/34; C08G 65/33396

(57) ABSTRACT

The present invention is related to a product for the resolution of racemic mixtures in which various separation processes of a salt composed of a PEGylated resolving agent and an enantiomer in a racemic mixture is utilized. Separation can be caused by temperature-dependent phase transformation of the said salt pair in aqueous or organic media as well as other methods used is separation of PEG such as salting out (e.g. addition of ammonium sulfate). The first cycle of racemic resolution by this method was shown to afford 85% optically pure amino acid from a 50:50 mixture of racemic L,D-amino acids esters. An additional cycle improved optical purity up to 95%.

### FIELD OF THE INVENTION

The present invention is related to a novel product used for the resolution of racemic mixtures by directed preferential precipitation of one enantiomer over the other using optically pure PEGylated (a)-hydroxy or amino acid in aqueous or organic media and by temperature-assisted phase transition of the diastereomeric salt pair or by its precipitation using various methods known to cause the precipitation of PEG.

### BACKGROUND OF THE INVENTION

Enantioselective synthesis has been both a source of fascination and challenge for chemists. The broad utility of chiral molecules in optically pure form as biologically active compounds, especially as pharmaceuticals and agrochemicals, as additives for modification of polymer properties and in electronic and optical devices explains the importance of chirality and methods of obtaining enantiopure compounds. Biochemicals such as proteins, enzymes, amino acids, carbohydrates, nucleosides, alkaloids and hormones are chiral compounds. In the pharmaceutical industries, approximately 50% of marketed drugs are chiral, and of these approximately 50% are mixtures of enantiomers rather than single isomer (see for example: Hutt A. J. et al, CNS Spectrums. 2002, 7, 14-22; Rentsch et al, Journal of Biochemical and Biophysical Methods, 2002, 54, pp. 1-9; Katzung et al. Basic and Clinical Pharmacology. 9^{th} Ed. New York: McGraw Hill; 2004, pp. 3-5). Tremendous advances have been made in asymmetric synthesis (see for example, Christmann, M. et al, Asymmetric Synthesis: The Essentials, 9^{th} Ed, Wiley: New York, 2007). However, the preparation of a racemic mixture followed by its resolution can still be a straightforward alternative, especially when both enantiomers are required or the undesired enantiomer can be easily converted to the desired one by simple chemical manipulation. Since the first separation of enantiomers by Louis Pasteur in 1848, a continuous search for new and efficient resolution procedures has been carried out. Classic resolution of enantiomers from a racemic mixture requires the introduction of a secondary chiral carbon in the enantiomer. This is usually achieved by salt formation, in which a resolving agent in the form of a chiral carboxylic acid or a chiral amine is used for diastereomeric salt pair formation. Covalent bond formation and complexes have also been used. Since diastereomers differ in their physical properties they are therefore separable. This is followed by the removal of the resolving agent, affording the optically pure enantiomer. The two determining factor in a successful resolution of enantiomers are the choice of the proper resolving agent and a suitable solvent for crystallization. Chiral carboxylic acids are valuable tools for chiral amines and chiral amines are used for resolution of enantiomeric carboxylic acids (see for example Fogassy et al. Org. Biomol. Chem., 2006, 4, 3011-3030). Interactions may include host-guest interactions, diastereomeric salts or diastereomeric covalent derivative formation. Therefore, besides the traditional well-known acid/base differentiating agents, the search for new useful compounds with chiral acids incorporated in their structures still continues.

PEGylation is the process of attaching the strands of the polyethylene glycol (PEG) to a molecule, most typically peptides, proteins, and antibody fragments that can improve the safety and efficiency of such therapeutics (see for example Veronese et al. Advanced drug delivery reviews 2002, 54, pp. 453-456). It produces alterations in the physiochemical properties of the PEGylated molecule including changes in conformation, electrostatic binding, hydrophobicity etc. These physical and chemical changes increase systemic retention of the therapeutic agent. Also, it can influence the binding affinity of the therapeutic moiety to the cell receptors and can alter the absorption and distribution patterns. Furthermore, PEG has been extensively used for protein precipitation in the production of various biopharmaceuticals such as monocolonal antibodies (mabs) (see for example K. C. Ingham, Methods Enzymol. 1984, 104, pp. 351-356; R. N; Haire, W. A. et al, Biopolymers, 1984, 23, pp. 2761-2779).

However, the use of PEG-assisted resolution of racemic mixtures has not been reported. We hereby report on novel PEGylated resolving agents and their utilization as highly efficient and innovative method for the resolution of racemic mixture with industrial applicability.

EP Pat. No. 0119804A1 explain a method for the resolution of D,L-α-amino acid such as D,L-leucine, D,L-valine and D,L-lysine using optically active α-phenylethanesulfonic acid as a resolving agent. Optical purity of resolved amino acids in this method is in the range of 60-94%. The less soluble diastereomeric salts in this method are passed through column packed with a strong acidic ion exchange resin to release the amino acid from the diastereomeric salt.

U.S. Pat. No. 4,322,548A provides a process for the resolution of racemic mandelic acid using alkyl esters of D or L phenyl glycine. In this method optically pure mandelic acid is obtained after several crystallizations in water.

U.S. Pat. No. 4,864,031 describes a process for the kinetic resolution of DL-racemic mixture of methionine hydrochloride, crystallizing it in the form of conglomerates from a supersaturated solution thereof by preferential crystallization in the presences of chiral crystal growth inhibitors that preclude or delay the nucleation of one enantiomorph, while leaving the opposite one unaffected, resulting in the preferential crystallization of the desired enantiomer.

U.S. Pat. No. 6,673,942B1 describes the resolution of DL-racemic mixture of DL-methionine hydrochloride, DL-glutamic acid hydrochloride, etc. which crystallize in the form of any conglomerate and some DL-racemic systems that exhibit various kind of crystal twinning, such as micro twinning by using poly-(N-methacryloyl-D-lysine) as a resolving agent. A preferential crystallization of one enantiomer from supersaturated solution in the presences of effective amount of poly-(N-methacryloyl-D-lysine) inhibits the crystallization of opposite enantiomer. In this process some loss of viscous DL-racemic mixture was observed.

U.S. Pat. No. 5,994,560 explains a process for resolving of racemic mixture by utilizing polymers with chiral moieties in their repeat units which exhibit critical solution temperature behavior in a suitable solvent.

Whitesell et.al. (J. Org. Chem., 1983, 48, 3548-3551) have reported the resolution of racemic alcohols by mandelic acid. Formation of the diastereomeric mixture of diesters of diols (containing approximately 20% of the meso isomer) was carried out with 2 equivalents of mandelic acid. Hydrolysis of the diester with aqueous potassium hydroxide afforded the diol in 95% yield and with an optical purity of 98%. However, this method caused partial racemization of the mandelic acid, thus preventing its reuse.

Rohani et.al. (Chirality, 2012, 24, 119-128) have reported the resolution of sertraline with mandelic acid by examining the weak intermolecular interactions (such as hydrogen bond and van der Waals interactions) and molecular packing difference in crystal structures of the resulting diastereomeric salts. Systematic examination of the intermolecular interactions and packing features in crystal structure of less soluble salt (1S, 4S)-sertraline.(R)-mandelic acid) elucidated the high resolution efficiency in the system.

PCT application WO2015029072A2 discloses a process for the resolution of racemic mixtures using high surface area core-shell functionalized polymer bead comprising a core of copolymer made from monomers selected from ethylene dimethacrylate and divinylbenzene and a shell which consists of monomers selected from glycidyl ethers of methacrylate and a chiral selector, chosen from tartaric acid derivatives and amino acids and its use in the resolution of (±)-terbutaline, (±)-Salbutamol.

Wan et.al. (Polym. Chem., 2014, 5, 1702,) reported the synthesis of a novel lysine bearing vinyl monomer, (S)-2-(tert-butoxycarbonylamino)-6-(40-vinylbenzamido) hexanoic acid for chiral resolution of racemic glutamic acid monohydrochloride. Polymerization was induced by azobisisobutyronitrile in the presence of 2-cyano-2-propyl dodecyl trithiocarbonate in dioxane. The polymers showed much better performance than the monomer. At a polymer concentration above 0.5 wt %, the R-enantiomer was obtained with an enantiomeric excess (ee %) over 97%.

Yashima et. al. (Nature, 1999, 399, 449) reports a series of optically active, cis-transoidal poly(phenylacetylene)s (PPAs) substituted with cinchona alkaloid residues pendants comprised of four pseudo-enantiomeric forms such as cinchonidine/cinchonine through an ester or amide-linkage for the resolution of racemates including N-Boc-amino acids. These polymers showed a preferred-handed helical conformation induced by the cinchona alkaloid pendants before being coated on macroporous silica gel to be used as chiral stationary phases (CSPs) in HPLC.

The above-noted processes, however, could not be operated easily since they require multistage and complicated operations for the resolution of racemic mixture. Furthermore, the prior art processes for the resolution of racemic mixture are deficient in that the purified product is obtained in a relatively low to moderate yields and optical purity which are not acceptable in industrial applications.

It would be desirable to have a relatively simple process with industrial applicability for the resolution of racemic mixtures.

### BRIEF DESCRIPTION OF THE INVENTION

In the present invention pure resolving agents such as (a)-hydroxy or (a)-amino acids, are condensed with activated polyethylene glycol-10000 (PEG-10000) through the amine or the hydroxyl function of the resolving agent to afford optically pure PEGylated-(α)-hydroxy or PEGylated-(α)-amino acids. The PEGylated resolving agent was dissolved in methanol and racemic mixture of DL- amino acid methyl ester such as DL-phenyl alanine methyl ester was added. The mixture is stirred for 4 h at room temperature and then cooled to 0-5° C. for 1 h. The resulting precipitate at was filtered 0-5° C. and washed with cold methanol. The resulting cake acidified by conc. HCl and again cooled to 0-5° C. and the precipitate was filtered. The yield and optical purity of this method was 80 and 85% respectively. Also, subjecting the enantiomerically enriched product to another cycle of resolution by this method increased optical purity from 85% to 90%.

### SUMMARY OF THE INVENTION

It is an object of the present invention to provide a novel resolving agent which can be used in an efficient process for the resolution of racemic mixtures such as DL-phenylalanine methyl ester using a PEGylated resolving agent by temperature dependent phase transition of the resulting salt pair between the PEGylated resolving agent and the corresponding enantiomer.

It is a further object of the present invention to provide a process which avoids the multistage and complicate operation and have an industrial application.

Accordingly, the present invention provides a process for preparing a PEGylated-(α)-hydroxy or (α)-amino acids with Formula 1 for racemic resolution of amino acids with Formula 2:

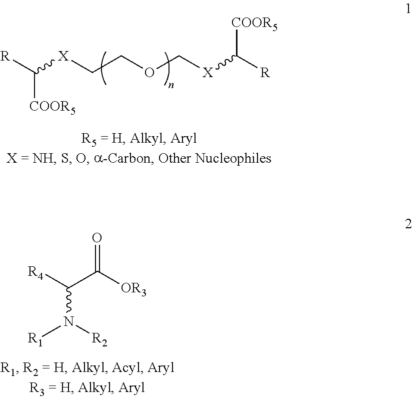

Starting from compounds of Formula 3 and 4:

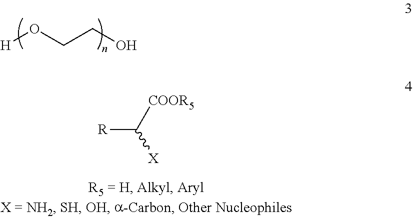

This comprises the steps of reacting 3 with activating reagent such as PCl₃, POCl₃, and preferably SOCl₂. Then reacting the activated PEG (5) with enantiomerically pure resolving agent containing a nucleophile such as a hydroxy, a mercapto an amino or any other potential nucleophile such as α-carbons to produce a compound of Formula 1 (X=N, O, S, etc.) as a resolving agent which can be used for the resolution of racemic mixture of Formula 2 .

Wherein R is selected from the group comprising phenyl, benzyl, a C₂-C₄ alkyl group, and the like; X is selected from the group comprising —O, —OH, SH, —NH, —NH₂, α-carbon, or other nucleophiles and R₁ and R₂ is selected from the group comprising, —H, Alkyl, Aryl and Acyl group; R₃ is selected from the group comprising —H, Alkyl, Aryl; R₄ is selected from the group comprising Alkyl, Aryl, benzyl; and R₅ is selected from the group comprising —H, Alkyl, Aryl, benzyl.

Alternatively, PEG could be substituted at one or both termini with a nucleophile such as N, S, O, α-carbon, etc. and the resulting chemically modified PEG reacted with a resolving agent carrying an electrophilic moiety.

### DETAILED DESCRIPTION OF THE INVENTION

The process of the invention is schematically represented as follows:

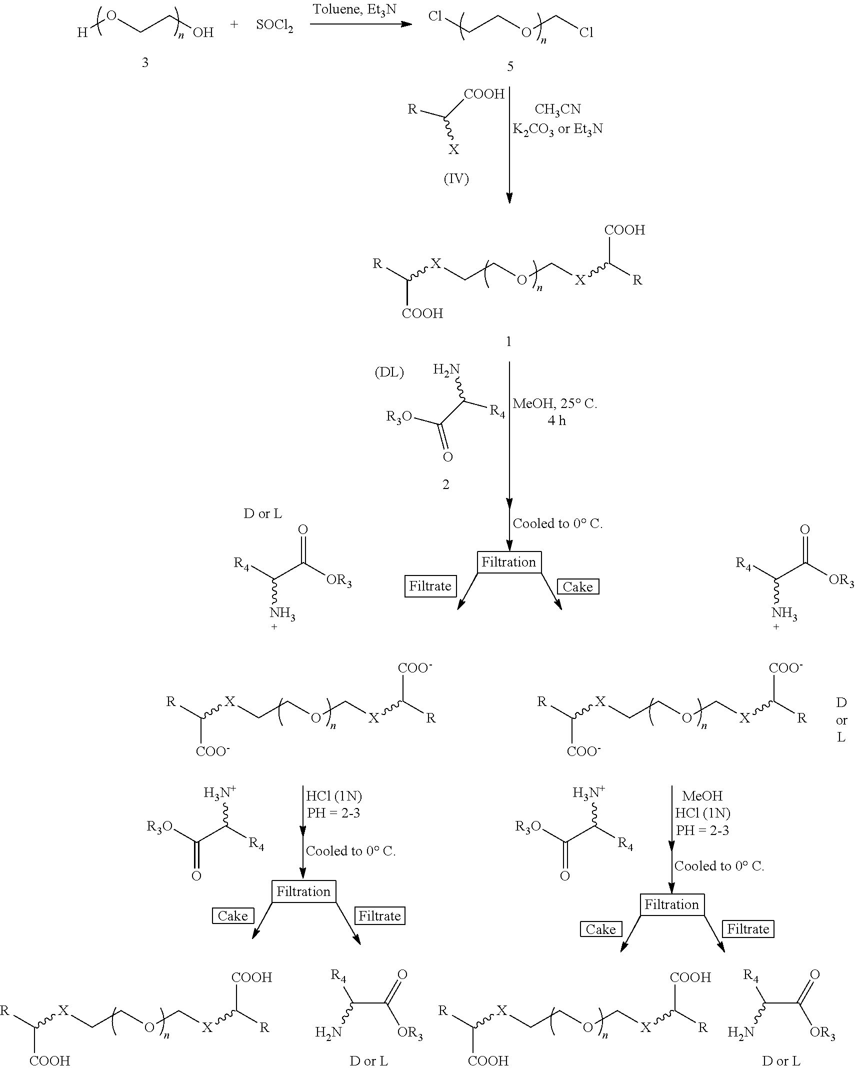

Preferably, the process for producing a compound of Formula 1 can be used to produce PEGylated (α)-hydroxy or (α)-amino acids or pharmaceutically acceptable salts thereof. Generally, PEGylated-α-hydroxy or amino acid may be prepared in two steps: (1) reacting the PEG-10000 with thionyl chloride and (2) reacting of activated PEG 5 with α-hydroxy or amino acids. In this process PEG reacted with at least 5 eq moles of thionyl chloride.

Typically, the reaction can be conducted at a temperature comprised between 15 to 25° C. and preferably at 20° C.

The base suitable for use in the process of producing a compound of Formula 1 is selected from the group comprising pyridine, Na₂CO₃ and preferably Et₃N or other bases used to neutralize the acid produced from the condensation reaction between 4 and activated PEG 5 for step of chlorination and K₂CO₃ for step of substitution.

The solvent suitable for use in the process of producing a compound of Formula 1 is selected from CH₂Cl₂, CHCl₃, THF, CH₃CN, DMF and preferably Toluene for step of chlorination and CH₃CN for step of substitution reaction.

The present invention further provide a process for racemic resolution of compound Formula 2 amino acid esters was added to enantiopure PEGylated-(α)-hydroxy or (α)-amino acids in methanol and upon cooling of the mixture to 0° C. a precipitate was formed. Diastereomeric salt formation of enantiopure PEGylated-(α)-hydroxy or (α)-amino acids with D or L-amino acids esters forms a pair of diastereomers that possess the same chemical formula, but have different physical properties. In this process, the molecules of opposite character (amine and acid) recognize each other by various interactions on the basis of their molecular structures and functional groups.

To effect the optical resolution of racemic amino acid esters, the amino acids esters is mixed with the PEGylated-(α)-hydroxy or amino acids and preferably 0.5 mole of PEGylated-(α)-hydroxy or amino acids is used per 2 mole of DL-amino acid esters.

The most preferred starting material of Formula 4 for the process of producing a compound of Formula 1 is a-hydroxy or (α)-amino acids in which R of Formula 4 is isopropyl or phenyl or other radicals known to men of art. In this embodiment, the product of Formula 1 is PEGylated-(R)-Mandelic acid or L-Valine respectively. It will of course be understood that the manner in which starting compound of Formula 3 is made is not particularly restricted as regards the process of making Formula 1 . In fact, any optically pure enantiomer with a nucleophile (to be condensed with a PEG that is activated with an electrophile) or an electrophile (to be condensed with a PEG that is activated with a nucleophile) can be used.

The most preferred racemic amino acid ester of Formula 2 is DL-amino acid ester in which R₃ of Formula 2 is methyl and R₄ is benzyl. In this embodiment, the DL-amino acid ester of Formula 2 is DL-phenylalanine methyl ester.

In general, it will be found that PEGylated-(R)-mandelic acid is better than of PEGylated-(L)-valine for resolving of DL-phenyl alanine methyl ester. After mixing the PEGylated-(R)-mandelic acid and the racemic phenylalanine methyl ester and stirring for 4h in room temperature, the resulting mixture is cooled to 0° C. and filtered. Optical purity in case of PEGylated-(R)-mandelic acid as a resolving agent was 85% and for PEGylated-(L)-valine was 74%. The separation of the diastereomeric complex can also be caused by precipitation of polyethylene glycol moiety of the diastereomeric salt using ammonium sulfate in water. Optical purity of e product obtained with PEGylated-(R)-mandelic acid as a resolving agent was 76%.

It has been suggested that the underlying principle for the precipitation of PEG as a result of addition of salts is due to interference in H-bond formation between the numerous oxygens of the polyether and water (Duong-Ly et. al. Methods Enzymol. 2014, 541, 85-94; Nathaniel et. al. J. Mol. Liq. 2008, 143-170).

The resolution medium can be water or alcohols or mixture thereof and suitable alcohols are methanol, ethanol, isopropanol, butanol and the like. In general, methanol is preferred resolution medium. The temperature ranges at which resolution is carried out are from 0° C. to 25° C.

Aspects of the present invention will be described with reference to the following examples which should not be considered to limit the scope of the invention.

### EXAMPLE 1

Preparation of Activated PEG (Chlorination of PEG)

PEG (10000) (50 g, 5 mmol) was dissolved in 500 ml of toluene and 100 ml of toluene was distilled from the solution to remove traces of moisture. After cooling to 35° C., freshly distilled anhydrous triethylamine (3.75 ml, 27 mmol) was added. Within 1 h freshly distilled thionyl chloride (1.5 ml, 21 mmol) was dissolved in 20 ml of dry toluene, and added dropwise at 35° C. to the mixture with continuous stirring under a dry nitrogen atmosphere. The mixture was refluxed for 1 h and triethylammonium chloride was removed by passing the hot solution through Celite. After 4h incubation at room temperature, the solution was heated to 50° C. and treated with 5 g of decolorizing carbon which was then removed by filtration over Celite. The filtrate was stored at 4° C. overnight, affording activated PEG which was filtered at 4° C. The solid product was further purified by dissolving in 2,5 1 of absolute ethanol at 60° C. and treating with 30 g of decolorizing carbon, followed by filtration over Celite. The ethanolic filtrate was stored overnight at 4° C. to recrystallize the product. The solid material was separated by filtration and washed with cold ethanol and then ether. After drying in a vacuum desiccator 49 g of a pale yellow product was obtained.

### EXAMPLE 2

Preparation of PEGylated-(R)-Mandelic acid

Mandelic acid (0.6 g, 4 mmol) was dissolved in 200 ml of acetonitrile, followed by to addition of K₂CO₃ (1.1 g, 8 mmol). Activated PEG (10000) (20 g, 2 mmol), prepared from example 1 was dissolved in 50 ml of acetonitrile and added dropwise with continuous stirring. Thereafter, the resulting pale yellow solution was stirred at reflux for 24 h. Then the reaction mixture was cooled to ambient temperature and adjusted to pH 2-3 using concentrated hydrochloric acid. The resulting solution treated with decolorizing charcoal and filtered over a layer of Celite. The resulting filtrate was evaporated under reduced pressure to obtain a solid which dissolved in methanol and the solution was stored at 4° C. overnight to crystallize the product. The solid material was separated by filtration and washed with cold methanol and then ether. After drying in a vacuum desiccator 15 g of a pale yellow product was obtained. The product was characterized by UV, NMR and IR spectra. ¹H-NMR (500 MHz, CDCl₃): δ 3.57 (s, n CH₂), 7.21-7.40 (m, 10H of Ar), 9.22 (bs, OH), 5.12 (s, CH). MS (EI) m/z: 152 (15), 107 (80), 79 (100).

### EXAMPLE 3

Preparation of PEGylated-(L)-Valine

L-valine (0.469 g, 4 mmol) was dissolved in 200 ml of acetonitrile. Trietylamine (1.11 ml, 8 mmol) was added to the reaction mixture. Activated PEG (10000) (20 g of, 2 mmol), prepared in example 1, was dissolved in 50 ml of acetonitrile and added dropwise to the reaction mixture with continuous stirring. Thereafter, the resulting yellow solution was stirred at room temperature for 24 h and then treated with decolorizing charcoal and filtered over a layer of Celite. The resulting filtrate was evaporated under reduced pressure to obtain a solid, which was then dissolved in methanol and the solution was stored at 4° C. overnight to crystallize the product. The solid material was separated by filtration and washed with cold methanol and then ether. After drying in a vacuum desiccator 18 g of a pale yellow product was obtained. The product was characterized by UV, NMR and IR spectra. ¹H-NMR (500 MHz, CDCl₃): δ 0.98 (m, 2CH₃), 2.63 (m, CH (CH₃)₂), 3.59 (s, n CH₂), 3.59 (m, CH(NH)). MS (EI) m/z: 116 (15), 72 (100), 55 (76), 43 (62).

### EXAMPLE 4

Resolution of Racemic Phenyl Alanine Methyl Ester with PEGylated-(R)-Mandelic Acid

PEGylated-(R)-mandelic acid (10 g, 1 mmol) was dissolved in 50 ml methanol followed by the addition of racemic mixture of phenyl alanine methyl ester (0.36 g, 2 mmol) and the mixture was stirred at room temperature for 12 h. It was then cooled to 0-5° C. and stirred for 1 h. A voluminous precipitate of white solids was formed, followed by the addition of 20 ml cold methanol. The slurry was filtered and the cake was washed with 10 ml cold methanol, resulting in white solids, consisting of optically impure PEGylated-(R)-mandelic acid.(L)-phenyl alanine methyl ester. The cake was then dissolved in 50 ml methanol and acidified with concentrated hydrochloric acid and cooled to 0-5° C. The white solid was filtered and washed with 10 ml cold methanol. The filtrate was evaporated under reduced pressure to dryness at 50° C. to yield 0.19 g (L)-phenyl alanine methyl ester hydrochloride (90%). Specific optical rotation was performed using sodium lamp D-line wavelength and shown +28 (EtOH, c=2) corresponding to 85% of theory.

### EXAMPLE 5

Resolution of Racemic Phenyl Alanine Methyl Ester with PEGylated-(L)-Valine

Using the method of example 4, PEGylated-(L)-Valine was used for resolving of racemic phenyl alanine methyl ester. In this example specific optical rotation shown +24 (EtOH, c=2) corresponding to 75% of theory.

It will be understood that the specification and example are illustrative, but not limiting to the present invention and that other embodiments within the spirit and scope of the invention will suggest themselves to those skilled in the art.

### EXAMPLE 6

Ammonium Sulfate-Assisted Resolution of Racemic Phenyl Alanine Methyl Ester with PEGylated-(L)-Mandelic Acid in Water

PEGylated-(R)-mandelic acid (10 g, 1 mmol), racemic phenyl alanine methyl ester (0.36 g, 2 mmol) were added to 50 ml water and the mixture was stirred at room temperature for 12 h. The resulting mixture was cooled to 0-5° C. and stirred for 1 h. Unlike Experiment 3.5, no precipitation occurred. Solid ammonium sulfate was added to the mixture. After 1 h a voluminous precipitate of a white solid occurred. The slurry was filtered and the cake was washed with 10 ml saturated ammonium sulfate, resulting in white solids consisting of optically impure PEGylated-(R)-mandelic acid.(L)-phenyl alanine methyl ester. The cake was dissolved in 50 ml water and acidified with concentrated hydrochloric acid and stirred for 1 h to liberate (L)-phenyl alanine methyl ester hydrochloride. Dry ammonium sulfate was then added to the resulting solution to precipitate PEGylated (R)-mandelic acid which was filtered and the cake was washed with 10 ml saturated ammonium sulfate. The filtrate was adjusted to pH 7 using sodium bicarbonate (1M) to neutralize (L)-phenyl alanine methyl ester. The resulting white precipitate was filtered, washed by 5 ml water and dried to afford 0.20 g of optically impure (D)-phenyl alanine methyl ester (yield: 93%). Specific optical rotation performed using sodium lamp D-line wavelength and shown +24 (EtOH, c=2) corresponding to 75% of theory.

### EXAMPLE 7

Improvement of Enantiomeric Excess by Additional Cycles of Resolution

A mixture of PEGylated-(R)-mandelic acid (5 g, 0.5 mmol), optically impure (L)-phenyl alanine methyl ester obtained from Experiment 3.5 (0.16 g, 0.9 mmol, optical purity 85%), and 25 ml methanol was stirred at room temperature for 12 h. The mixture was cooled to 0-5° C. and stirred for 1 h. A voluminous white precipitate formed, followed by the addition of 20 ml cold methanol. The slurry was filtered, and washed with 10 mL cold methanol. The resulting white solid was dissolved in 50 ml methanol and acidified with concentrated hydrochloric acid pH=2-3 and cooled to 0-5. The white solid was filtered and washed with 10 ml cold methanol. The filtrate was evaporated to dryness at 50° C. under vacuum to yield 0.18 g (L)-phenyl alanine methyl ester hydrochloride (yield: 92%). Specific optical rotation performed using sodium lamp D-line wavelength and shown +32 (EtOH, c=2) corresponding to 95% of theory.

What is claimed is:

1. A resolving agent that can be used for the resolution of racemic mixtures and is composed of a chemically activated linear or branched polyethylene glycol (PEG) from molecular weights of 400 to 4 million in which the PEG is activated in one terminus or many termini and condensed with to a pure stereoisomer and in which the PEG termini carries an nucleophile(s) to be condensed with a pure isomer carrying an electrophile or in which the PEG termini carries an electrophile(s) to be condensed with a pure isomer carrying an nucleophile.

2. Utilizing the said PEGylated resolving agent in an aqueous or organic medium for diastereomeric salt pair formation between the said PEGylated resolving agent and a mixture of enantiomers by cooling the resulting mixture or by the addition of agents known to result in the precipitation of polyethylene glycol.

3. A resolving agent of claim 1 composed of various known polyethylene glycols such as linear or branched (PEG), attached to a pure stereoisomer (SI), including those traditionally used for resolution of racemic mixtures such as mandelic acid, brucine, strychnine, (D)- or (L)-tartaric acid, valine or any stereochemicaly pure compound capable of forming diastereomeric salts with racemic mixtures of enantiomers, via a linkage X formed between a chemically activated PEG at one terminus or many termini, and in which chemical activation depends on the nature of SI so that when SI is a nucleophile the said chemical activation of PEG involves the attachment of an electrophile to PEG terminus (or termini) and when SI is an electrophile the said chemical activation of PEG involves the attachment of a nucleophile to PEG terminus (or termini)—as shown below as A:
SI-X-PEG-X-SI A

4. A resolving agent of claim 1 composed of various known polyethylene glycols such as linear or branched (PEG), attached to a pure stereoisomer (SI) carrying an electrophilic moiety. The stereoisomer is chosen, but not limited to, the traditional resolving agents such as mandelic acid, brucine, strychnine, (D)- or (L)-tartaric acid, valine or any stereochemicaly pure compound capable of forming diastereomeric salts with racemic mixtures of enantiomers, via a linkage formed between a chemically activated PEG at one terminus or many termini (Y-PEG-Y, Y=N, S, O, α-carbon, etc.) used for condensation with a resolving agent carrying an electrophilic moiety as shown below as B.
SI-Y-PEG-Y-SI B

5. A resolving agent of claim 3 in which the PEG used may be substituted with group comprising of phenyl, benzyl, naphthyl, a C₂-Cₙ linear or branched alkyl group that may carry inert functional groups such as nitrile, ethers, amide, ester, olefin, alkyne, and the like.

6. A resolving agent of claim 3 in which X (Nucleophile on the resolving agent) or Y (Nucleophile on PEG) may be —O, —OH, SH, —NH, —NH₂ and other nucleophiles such as α-carbons that can form carbanions, etc.

7. A resolving agent of claim 1 in which a stereochemically pure amino acid, alkaloids, nucleosides, or any optically pure compound or a derivative thereof is attached to PEG and the resulting resolving agent (e.g. SI-X-PEG-X-SI or SI-Y-PEG-Y-SI vide supra) is used for the resolution of racemic amino acid esters or any other racemic mixture in aqueous or organic solvents and recovering the D-amino acid esters and L-amino acid ester or the enantiomers of the racemic mixture as shown below as 1 and 2.

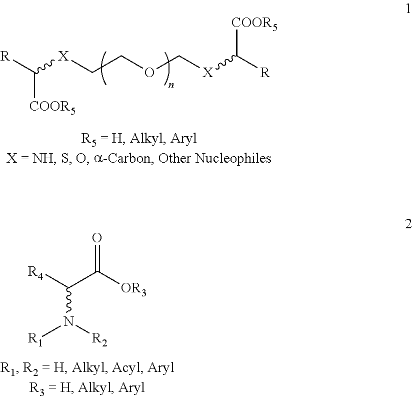

8. A process for the separating of enantimerically pure compounds from their racemic mixtures in which a resolving agent of claim 1 is used and by cooling the final resolution solution and filtration or centrifugation of the precipitate, followed by the release of the desired enantiomer by treatment with acid or base.

9. A process for the separation of enantimerically pure compounds from there racemic mixture in which a resolving agent of claim 1 is used and by the addition of various inorganic and organic chemicals that are known to cause precipitation of PEG.

10. A process for the separation of enantiomerically pure compounds from their racemic mixtures (compound 2) in which the SI in claim 2 is D- or L-phenyl alanine methyl ester.

11. A process for the separation of enantoimerically pure compounds from their racemic mixtures in which the SI is claim 2 is (R)- or (L)-mandelic acid.

12. A process for the separation of enantiomerically pure compounds from their racemic mixtures in which the SI in claim 2 is enantiopure-(α)-hydroxy acids.

13. A process for the separation of enantiomerically pure compounds from their racemic mixtures in which the SI in claim 2 is (L)- or (D)-valine.

14. A process for the separation of enantiomerically pure compounds from their racemic mixtures in according to claim 1 in which the resolving agent is used in an amount of 0.25 to 0.5 mole per mole of racemic phenyl alanine methyl ester to be resolved.

15. A process of claim 1 in which the base suitable for use in the process for synthesis of a compound of formula A is selected from the group comprising pyridine, diisopropylethyl amine, triethylamine, Na₂CO₃, Cs₂CO₃ and K₂CO₃.

16. The process defined in claim 2, wherein MeOH, EtOH, and H₂O is used as solvent for the resolution of enantiomers.

17. Process as claimed in claim 2 wherein said resolution is carried out at from 0° C. to 25° C.
